(19) United States
(12) Patent Application Publication
(10) Pub. No.: US 2010/0015321 A1
(43) Pub. Date: Jan. 21, 2010

(54) SCAFFOLD TREATMENT - DEVICE AND METHOD

(75) Inventors: Justin John Cooper-White, Queensland (AU); Tristan Croll, Queensland (AU)

Correspondence Address: WOLF GREENFIELD & SACKS, P.C., 600 ATLANTIC AVENUE, BOSTON, MA, 02210-2206, US

(73) Assignee: The University of Queensland, Queensland (AU)

(21) Appl. No.: 12/310,125

(22) PCT Filed: Aug. 10, 2007
(86) PCT No.: PCT/AU2007/001130

§ 371 Date: Feb. 11, 2009

(30) Foreign Application Priority Data
Aug. 11, 2006 (AU) 2006904384

(51) Int. Cl. B05D 3/00; B05C 5/02

(52) U.S. Cl. 427/2.1; 118/300

(57) ABSTRACT

An apparatus for treating porous polymeric scaffolds including an enclosure; a support for at least one porous polymeric scaffold, and at least one application outlets within the enclosure for applying a liquid agent onto the top surface of the at least one porous polymeric scaffold; each porous polymeric scaffold being supported on a drained platform above a liquid catchment area within the enclosure and having at least one application outlet directed at its top surface

### FIELD OF THE INVENTION

This invention relates to an apparatus and method of applying a liquid to a porous polymeric substrate. In particular, the porous polymeric substrates is used as a scaffold for tissue engineering.

### BACKGROUND OF THE INVENTION

Tissue engineering promises to provide the supply of biological implants required by surgeons for the repair or the replacement of a variety of native tissues that are weakened, damaged or obstructed by trauma or disease. Historically these implants have been either homografts, synthetic grafts made from synthetic materials or fresh or fixed tissue grafts.

Due to the inadequacies of these currently available synthetic and biological grafts as well as the cost and limited supply of homografts, tissue engineered grafts are being developed in which synthetic scaffold material is seeded and cultured, in vitro with cells.

A tissue engineering scaffold material should ideally satisfy a number of key physical requirements. It must either be fully degradable to products which may be eliminated from the body or remain stable indefinitely. Another requirement is that the surface of the synthetic scaffold material should be biocompatible for the attachment and cultivation of cells. Additionally, it should be possible to manufacture the material and structures which are highly porous with all pores accessible. Critically the physical properties of the scaffolds produced from the materials should be such that they are not damaged by normal day to day movement and pressures and conversely do not damage the surrounding tissue.

The most common tissue engineering solid structures and materials have been the poly (lactic-co-glycolic acid) (PLGA) family mainly due to their long history of medical use and FDA-approved status. Other commonly used polymer classes include polyanhydrides, polycarbonates, polycaprolatones and polyfumrates. In general, these polymers degrade to various carboxylic acids which are readily metabolised and/or excreted.

While the high mechanical strength, processability and degradability of these polymers make them attractive materials, their surface properties are a significant problem.

To improve the biocompatibility of these materials, surface modification and coatings are needed to chemically functionalise these largely inert polymers for use as scaffolds. The modification and coating of these materials often requires several processing steps to accomplish the required material coating. For example, surface modification typically includes a surface activation step followed by the coupling of the desired biomolecule. As the active surface of the scaffold includes the pores, access to the pores during the surface activation and coating steps is essential. Furthermore, the manufacture and use of modified surface substrates require a sterilisation method to finalise the product for use. However, conventional sterilisation methods such as steam, radiation and ethylene oxide negatively impacts the physical properties of scaffolds. As with the modification and coating methods, the sterilisation method is also required to access the pores of the scaffold without affecting the stability of the modified surface and the scaffold.

According to FDA guidelines on current good manufacturing practice, sterilisation prior to further processing under aseptic conditions provides an acceptable level of sterility provided that proper controls are in place. As mentioned above, adequate sterilisation of porous scaffolds by traditional methods such as gamma radiation, ethylene oxide, electron beam and ultra violet radiation all lead to unacceptable levels of degradation of bio-degradable scaffolds such as polyesters e.g. PLGA.

Hence, there is a need to be able to treat the surfaces of the highly porous polymeric materials to be used as scaffolds.

### SUMMARY OF THE INVENTION

In order to improve the usability of scaffold material of a porous polymeric nature, the invention provides an apparatus for treating a porous polymeric scaffold including an enclosure; a support for at least one porous polymeric scaffold, and at least one application outlet within the enclosure for applying a liquid agent onto the top of the at least one porous polymeric scaffold; each porous polymeric scaffold having at least one application outlet directed at its upper surface.

The apparatus according to this aspect of the invention may be used to apply a liquid agent which contains a surface treatment agent for modifying or coating the pores of the scaffold material. The liquid agent may contain one or more biomolecules for attaching to the pore surfaces of the scaffold or a sterilisation agent which has little degradative effect on the scaffold material.

The porous polymeric scaffold used in the apparatus may have been treated prior to use in the apparatus of the present invention to wet the pore surfaces within the scaffold material. The scaffold material which is preferably supported on a drained platform is preferably supported above a liquid catchment area within the enclosure. The drained platform which is preferably apertured but may be simply contoured to prevent pooling of liquid agent around the base of the scaffold. The liquid catchment area is below the lower surface of the scaffold enabling liquid agent applied to the top surface of the scaffold material to flow through the scaffold into the catchment area only under the influence of gravity and capillary forces within the scaffold.

In one preferred form of the invention the liquid agent is supplied to a manifold within the enclosure. The manifold may communicate with at least one application outlet within the enclosure to supply the liquid agent to the porous polymeric scaffold.

In an alternative form of the invention, the liquid agent may be supplied to the enclosure by a plurality of conduits corresponding to the number of application outlets within the enclosure.

In another aspect of the invention, there is provided a method of treating the pores of a porous polymeric scaffold including the steps of wetting the pores of a porous polymeric scaffold and applying a liquid agent to the upper surface of the scaffold, the liquid agent passing through the pores of the polymer scaffold into a region below the scaffold.

In the method, the pores of the scaffold are subjected to a wetting process in which the pores are either soaked in a wetting solution or the wetting solution is otherwise forced into the pores by pressure or vacuum. Hence when placed onto the apertured support, the wetting solution is able to drain or begins to drain from the scaffold prior to the application of the liquid agent.

It is preferable that substantially all of the sides of the scaffold are physically unrestrained during the method of the invention. It is believed that by providing a form of side restraint on the scaffold that channelling may possibly occur between the restraint and the side of the scaffold resulting in inadequate flow through the pores of the scaffold. It is intended that all of the flow of liquid agent occur into the top surface of the scaffold through the pores, exiting the base of the scaffold under the action of gravity and capillary forces.

In a preferred form of this invention, the method is carried out in an enclosure containing at least one wetted porous polymeric scaffold, the enclosure having at least one outlet for delivery of liquid agent to the top surface of each scaffold.

The liquid agent may be a sterilizing agent such that this aspect of the invention provides a method of sterilizing the porous polymeric scaffold. Alternatively the liquid agent may contain a biomolecule for attaching to the pores of the scaffold to chemically functionalise the porous surfaces of the scaffold.

Sterilization of the apparatus, prior to treatment of the scaffold, occurs by initially adding a sterilizing agent as the treatment solution to sterilize the liquid contacting surfaces. The non-liquid contacting surfaces within the enclosure are preferably sterilized by supplying a sterilizing gas or aerosols of the sterilizing agent to the enclosure via a gas inlet into the enclosure.

The apparatus preferably includes filters on all inlets and outlets to and from the enclosure. The filters are designed as one way filters to allow flow of fluid in one direction only thereby effectively creating a sterile barrier around the enclosure. This allows scaffold placed in the enclosure to be sterilized and remain sterile during treatment.

Further features, objects and advantages of the invention will become more apparent from the following description of the drawings and preferred embodiment.

### BRIEF DESCRIPTION OF THE DRAWINGS

FIG. 1 is a schematic diagram of an embodiment of the invention, and

FIG. 2 is a schematic diagram of supporting apparatus used in an automated treatment system.

### DETAILED DESCRIPTION OF THE EMBODIMENTS

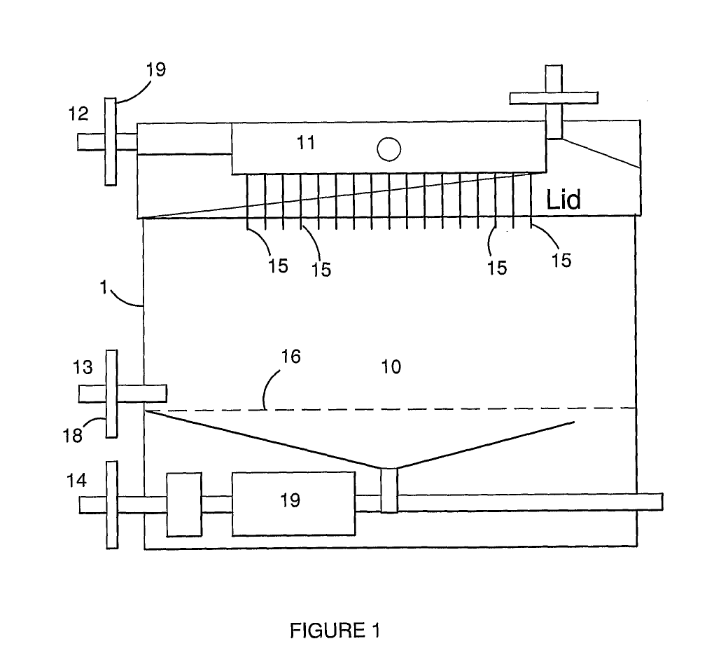

Referring to FIG. 1, the invention includes an enclosure 1 having a sealing lid 1A which contains a liquid agent distributor 11. The lid 1A is provided with an inlet conduit 12 for the entry of the liquid agent into the liquid agent distributor 11. The liquid agent distributor illustrated includes a manifold 11A provided with a plurality of application outlets 15 into the treatment region 11B of the enclosure. While the invention has been illustrated using a manifold 11A to introduce liquid agent to the plurality of application outlets 15 in the treatment region 11B of the enclosure 1.

The manifold 11A may be substituted for a plurality of individual liquid agent lines entering the lid 1A. The number of individual lines can be matched to the number of outlets or a single line may supply agent to a number of application outlets within the enclosure 11. In this arrangement the distribution of liquid to the individual lines supplying the plurality of application outlets takes place outside of the enclosure 1.

The enclosure 1 is further provided with a support or platform 10 for supporting porous scaffolds (not shown). The platform 10 is preferably indexed to enable the porous scaffolds to be precisely positioned beneath at least one application outlet 15. This enables the scaffold to be always positioned in the correct place on the platform to receive liquid agent from a single application outlet onto the top surface of the scaffold. This is a highly desirable requirement for operation of the invention.

The platform 10 has apertures 16 to enable treatment solution/liquid agent which is passed through the scaffold to drain from the scaffold to a catchment area 17 below the scaffold support 10.

For efficient operation of the apparatus of the invention, the liquid agent should not be allowed to pool around the scaffold above the lower surface of the scaffold to ensure that there is sufficient driving forces (capillary and gravity) for the liquid agent to pass through the porous scaffold. The liquid agent collected in the catchment area 17 is removed through a liquid outlet 14. The liquid removed through the outlet 14 may be discarded or recycled to inlet 12, if appropriate.

A positive pressure relative to atmospheric pressure is applied to the enclosure via a gas inlet 13. The pressure within the enclosure is preferably slightly above atmospheric to prevent contamination of the treatment area.

Essentially the scaffold is sealed within the chamber and subjected to either liquid treatment or sterilisation depending on the liquid agent introduced into the apparatus.

Treatment solutions which can be usefully used with the invention include: solutions for functionalization of the scaffold surface; solvent/non solvent mixtures for loading functional polymers, such as polylysine and polyaspartic acid for surface entrapment modification; polyelectrolytes for layer-by-layer deposition; biologically functional molecules; passivating molecules and/or sterilizing agents.

By way of example simple functionalization treatments for polyesters may include aqueous, dilute bases such as. primary amine-containing molecules e.g. 0.01-1 M sodium hydroxide solution with 0.05-1 M ethylene diamine (or other polyamines such as diethylene triamine, triethylene tetramine, N-aminoethyl-propanediamine etc.); and compounds having primary amines groups in combination with an additional functional group, such as glycine (carboxylic acid), cysteine (thiol and carboxylic acid), cysteamine (thiol) or the like.

Polyelectrolytes for layer-by-layer desposition may be selected from;
a) SYNTHETIC: such as polyethylenimide, poly (styrenesulfonate) and poly (acrylic acid);
b) SEMI-SYNTHETIC: such as polylysine or ornithine (L- and/or D-isomers), polyaspartic or glutamic acid (L- and/or D), chitosan, chitosan sulphate, DNA and gelatin; or
c) BIOLOGICAL: such as hyaluronic acid, heparin, heparan sulphate, dermatan sulphate, pectin, alginate and most of the collagens.

Biologically functional molecules may include extracellular matrix (ECM) molecules such as fibronectin, vitronectin, collagens, laminins, heparan sulphate proteoglycans and the like or growth factors such as the fibroblast growth factors (FGFs), endothelial growth factors (EGFs) and platelet derived growth factor (PDGF).

Passivating molecules may include variously functionalized poly(ethylene oxide) (PEO), poly(hydroxyethyl methacrylate) (PHEMA), alginate and hyaluronic acid.

Suitable sterilizing agents include all FDA-cleared sterilants for device sterilization (a list of such sterilants can be found at www.fda.gov/cdrh/ode/germiab.html).

For liquid contacting areas, the preferred sterilizing agents may be selected from aqueous solutions containing 0.1-5% peroxycarboxylic acids (peroxyformic, -acetic, -propanoic, -benzoic and the like, with or without the presence of alcohols, such as ethanol or isopropanol; aqueous solutions containing 0.1-10% of an aldehyde, such as formaldehyde, paraformaldehyde or glutaraldehyde, with or without alcohols; or aqueous solutions containing >˜5% hydrogen peroxide . . . . The apparatus may be placed under mild heating conditions. It has been found that mild heating in the range of 30-60° C. may lead to a drastic reduction the time to sterilize the apparatus or scaffold. It is preferable that if the sterilizing agent comprises peroxycarboxylic acids that the peroxycarboxylic acid is selected from formic acid or acetic acid, as they do not adversely affect the active cellular uptake.

For non-liquid contacting areas, the preferred sterilizing agents include gas containing 0.001-1% by volume of a peroxycarboxylic acid, or aerosol of any of the above sterilizing agents or solutions, potentially with an added surfactant to ensure thorough wetting of all surfaces.

All solutions and gasses entering the apparatus of the invention enter via sterilizing grade filters 18, 19 located on the gas inlets and liquid conduits or inlets, 13 & 12 respectively while the enclosure is maintained under slightly positive pressure via a sterilizing filter. The liquid agent or treatment solution enters through inlet 12 into either a manifold 11 or through a series of individual lines and the scaffolds in the treatment region of the enclosure are subjected to the treatment solutions. The scaffolds sit on a platform 10 which may be a mesh tray allowing solution to pass therethrough and discharged liquid is constantly withdrawn from a catchment region below the scaffold platform by a peristaltic pump 19A.

Prior to being placed into the apparatus of the invention, the scaffold(s) are preferably soaked for a period of time in a wetting solution in order to thoroughly wet all of the pores in the scaffold. The wetting solution within the scaffold may be allowed to partially drain before the application of a treatment solution within the enclosure. Once the treatment solution is applied to the top surface of the scaffold, capillary forces and gravity draw the treatment solution through the pores of the scaffold exiting through the base of the scaffold.

In performing the method of the invention it is preferable that the peripheral sides of the scaffold are unrestrained to prevent the problem of channelling between the scaffold and the restraint thereby offering a flow path of lower resistance and seriously affecting the flow of treatment solutions through the scaffold.

An additional example of the use of the invention will now be described.

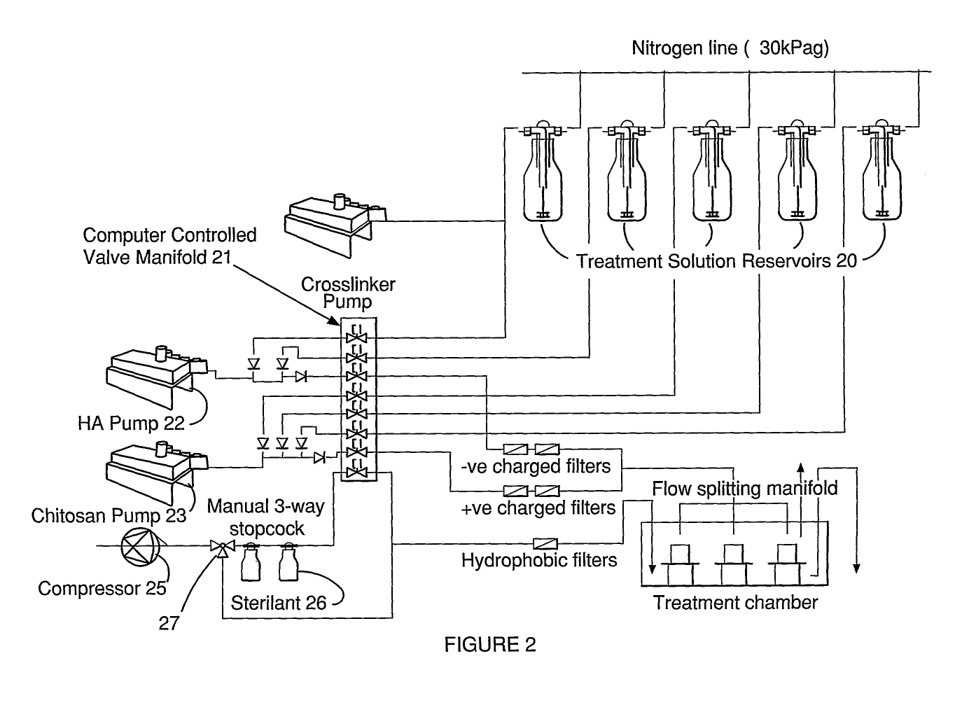

FIG. 2 shows a schematic diagram of an automated treatment system, which provides for both sterilization and layer by layer treatment of a scaffold.

Prior to the layer by layer treatment, the scaffolds and chamber are sterilized by simultaneously pumping wet sterilant onto the scaffolds while flushing the chamber with air pumped from the compressor 25 through the sterilant bottle 26. Once the sterilisation is complete the chamber is maintained under positive pressure by switching the manual three way stop cock 27 to by-pass the vapour sterilants.

Treatment solutions are stored in treatment solution reservoirs 20 which are pressurised to approximately 30 kpa with an inert gas such as nitrogen. These solutions are drawn from the treatment solution reservoirs 20 via a computer controlled valve manifold 21 into the desired pump. In this embodiment which uses hyaluronic acid (HA) in the layer by layer treatment, a cross linker solution in dimethylformamide is added to the flow at a volumetric ratio of 1 to 100 during the filling step. Separate syringe pumps 22, 23 are used for positive and negative charged polymer solutions to prevent cross-contamination. When ready the respective layer treatment solutions are pumped out again under the control of the value manifold and through 0.2 μm filters and into the inlet of the apparatus 1. The liquid agent is then flow split by manifold 11 and applied to the scaffold as described above.

The volume of each treatment step was set to approximately four times the volume of scaffold present, while rinses were approximately six times the scaffold volume. Flow rates were set so that this solution flowed continually through the scaffolds over 10 minutes (3 minutes per rinse). Polyethylenimine concentration was set at 20 mg/mL, and the scaffolds were aminolysed for 2 hours (under static conditions after the first 10 minutes). HA and chitosan concentrations were approximately 250 μg/mL. The crosslinking agent stock solution was 50 mg/mL EDAC [1-ethyl-3-(3-dimethylaminoprophy)-carbodiimide/70-100 mg/mL NHS (N-hydroxysuccinimide) in DMF (dimethyl/formamide), and was added at a rate of 1:100 to the HA solution during filling. The HA solution for each step was filled at the same time as the chitosan solution for the previous step, allowing approx. 13 minutes to activate.

Using the automated system described above, a set of 24 scaffolds (15 mm diameter, 1 mm thick, 3 stacks of 8) was coated with 101 layers (501/2 bilayers) of HA and chitosan, over a period of approximately 26 hours. The first chitosan layer (layer 2) was made from far red fluorescent BODIPY 630-labelled chitosan. One scaffold was taken from a position in the middle of a stack, and further coated with a final layer of green fluorescent Alexa Fluor 514 labelled chitosan. A portion of this scaffold was then sectioned approximately halfway up its height using a double-edged razor blade, and mounted for confocal microscopy.

Upon reviewing confocal projections of the sectional portion of the scaffold, it was seen that all pore surfaces appeared well covered. Additionally strong far red fluorescence was observed throughout the majority of the scaffold indicating that the chitosan second layer had apparently penetrated the majority of the micropores and conformed to the surface.

While the invention has been described above for the application of HA and chitosan to the pore surfaces of scaffold, it will be appreciated by a person skilled in the art that other treatment solutions could also be applied by the method and apparatus of the invention.

It will be understood that the invention disclosed and defined in this specification extends to all alternative combinations of two or more of the individual features mentioned or evident from the text or drawings. All of these different combinations constitute various alternative aspects of the invention.

1. An apparatus for treating porous polymeric scaffolds including an enclosure; a support for at least one porous polymeric scaffold, and at least one application outlets within the enclosure for applying a liquid agent onto the top surface of the at least one porous polymeric scaffold; a drained platform for supporting each porous polymeric scaffold above a liquid catchment area within the enclosure and having such that the at least one application outlet is directed at the top surface of at least one porous polymeric scaffold.

2. The apparatus of claim 1 wherein the liquid agent applied to the top surface of each scaffold flows under the influence of gravity and capillary forces only through the scaffold to the liquid catchment area beneath the drained platform.

3. The apparatus of claim 1 wherein the liquid agent contains a surface treatment agent for modifying or coating the pores of the scaffold material.

4. The apparatus of claim 2 where the liquid agent contains one or more biomolecules for attaching to the pore surfaces of the scaffold or a sterilisation agent.

5. The apparatus of claim 4 wherein the drained platform is apertured or contoured to prevent pooling of liquid agent around the base of the scaffold.

6. The apparatus of claim 1 wherein the liquid agent is supplied to a manifold within the enclosure, the manifold communicating with at least one application outlet within the enclosure to supply the liquid agent to the porous polymeric scaffold.

7. The apparatus of claim 1 wherein the liquid agent is supplied to the enclosure by a plurality of conduits corresponding to the number of application outlets within the enclosure.

8. The apparatus of claim 1 wherein the drained platform is indexed to provide a precise location for each scaffold beneath at least one application outlet.

9. The apparatus of claim 1 or 8 wherein the support for the scaffolds or enclosure do not provide any physical restraint around the sides of the scaffolds.

10. Method of treating the pores of a porous polymeric scaffold including the steps of treating the pores of a porous polymeric scaffold and applying a liquid agent to the upper surface of the scaffold, the liquid agent passing through the pores of the polymer scaffold into a region below the scaffold.

11. The method of claim 10 wherein the pores of the scaffold are subjected to a wetting process in which the pores are either soaked in a wetting solution or the wetting solution is otherwise forced into the pores by pressure or vacuum.

12. The method of claim 10 or 11 wherein the method is carried out in an enclosure containing at least one wetted porous polymeric scaffold, the enclosure having at least one outlet for delivery of liquid agent to the top surface of each scaffold.

13. The method of claim 9 wherein the liquid agent delivered to the top surface of the polymeric scaffold flows through the scaffold under the influence of gravitational and capillary forces only into the region below the scaffold.

14. The method of claim 10 wherein the sides of the scaffold are unrestrained.

15. The method of claim 12 wherein the liquid agent is a sterilizing agent or contains a biomolecule for attaching to the pores of the scaffold to chemically functionalise the porous surfaces of the scaffold.

16. The method of claim 10 wherein more than one liquid agent is applied sequentially to the scaffold.
